(19) United States
(12) Patent Application Publication
(10) Pub. No.: US 2022/0304867 A1
(43) Pub. Date: Sep. 29, 2022

(54) MULTI-PIECE ABSORBENT ARTICLES AND ARRAYS THEREOF

(71) Applicant: The Procter & Gamble Company, Cincinnati, OH (US)

(72) Inventors: Sara Lyn GIOVANNI, Cincinnati, OH (US); Udo Friedel SCHOENBORN, Bad Soden (DE); Tonya Mae Sheldon WEST, Loveland, OH (US)

(21) Appl. No.: 17/699,299

(22) Filed: Mar. 21, 2022

Related U.S. Application Data
(60) Provisional application No. 63/164,627, filed on Mar. 23, 2021.

(51) Int. Cl. A61F 13/551; A61F 13/505

(52) U.S. Cl. CPC A61F 13/5511; A61F 13/505

(57) ABSTRACT

An array of absorbent articles includes a first absorbent insert, a second absorbent insert and one or more reusable outer covers configured to support the first absorbent insert to form a first absorbent article and/or configured to support the second absorbent insert to form a second absorbent article. The first absorbent insert has a first capacity and a first dry mass and the second absorbent insert has a second capacity and a second dry mass. The second dry mass is no more than 15% greater than the first dry mass and the second absorbent capacity is at least 15% greater than the first absorbent capacity.

### CROSS REFERENCE TO RELATED APPLICATIONS

This application claims the benefit of U.S. Provisional Application No. 63/164,627, filed Mar. 23, 2021, which is incorporated by reference herein in its entirety.

### FIELD

The present invention relates generally to the field of wearable absorbent articles having absorbent inserts and reusable outer covers.

### BACKGROUND

Absorbent articles (e.g., diapers, adult incontinence articles, feminine hygiene pads) offer the benefit of receiving and containing urine and/or other bodily exudates (e.g., feces, menses, mixture of feces and urine, mixture of menses and urine, etc.). It has been proposed to manufacture two-piece absorbent articles with a reusable outer cover and a detachable absorbent insert that may be reusable or disposable. In this way, the insert can be made with different materials to enhance performance and less energy may be consumed as the insert can be separately laundered. Further, where disposable, the inserts may be made with materials known to provide even more superior performance while minimizing the amount of waste as the whole article need not be disposed.

Despite several designs of a two-piece absorbent article, the designs still present some disadvantages. In particular, the use of more materials (e.g., absorbent materials, acquisition materials, padding) can result in more environmental waste. On the other, these same materials may be critical for sufficient performance. Thus, balancing material amounts and performance can prove challenging. Further, in some configurations, two-piece articles are provided as a one-size-fits-all to further reduce waste and production costs associated with multiple sized articles. Balancing performance and material usage across a variety of wearers' sizes and capacity needs is especially difficult.

Therefore, there is a need for improved two-piece articles that balance performance with material usage. There is also a need to strike said balance with minimum complexity for the consumer. Likewise, there is a need to create suitable products while minimizing complexity and/or cost in production.

### SUMMARY

The invention comprises the features of the independent claims herein. An array of absorbent articles comprises a first absorbent insert comprising a first absorbent capacity and a first dry mass. The array comprises a second absorbent insert comprising a second absorbent capacity and a second dry mass. The second dry mass is no more than 15% greater than the first dry mass and the second absorbent capacity is at least 15% greater than the first absorbent capacity. The array comprises one or more reusable outer covers configured to support the first absorbent insert to form a first absorbent article and/or configured to support the second absorbent insert to form a second absorbent article.

### BRIEF DESCRIPTION OF THE DRAWINGS

FIG. 1 is a perspective view of a wearable absorbent article as it might appear being worn by a wearer about the lower torso;

FIG. 2 is a plan view of an array of absorbent articles;

FIG. 3 is a perspective view of a package of absorbent articles;

FIG. 4 is a perspective view of a plurality of packages of absorbent articles;

FIG. 5 is a plan view of an outer cover opened and laid flat, inner surface facing the viewer;

FIG. 6 is a plan view of an insert opened and laid flat, outer (garment-facing) surface facing the viewer;

FIG. 7 is a perspective view of a disposable absorbent insert shown apart from an outer cover, as it might appear in a free-standing, relaxed state;

FIG. 8 is a plan view of a disposable absorbent insert shown stretched out and laid flat, wearer-facing surfaces facing the viewer;

FIG. 9 is a cross sectional view of an example of an insert such as shown in FIG. 8, taken at line 9-9 in FIG. 8;

FIGS. 10A-10B are schematic lateral cross sectional views of examples of inserts having a dual leg gasketing system, taken through a lateral axis of the exemplary inserts; and

FIGS. 11A-11D are exploded cross sectional views of exemplary absorbent inserts taken along their respective lateral axes.

### DETAILED DESCRIPTION

“Absorbent article” means a device that absorbs and contains body exudates and, more specifically, devices that are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, and the like.

“Absorbent insert” and “insert” mean a component of a wearable absorbent article that is adapted to contain and/or absorb urine, feces, menses or any combination thereof, and is adapted to be installable and removable as a modular unit, from an outer cover. Herein, an absorbent insert may also be referred to as an “absorbent assembly”. The terms “absorbent insert,” “insert” and “absorbent assembly” may be used interchangeably herein.

“Brand name” means a single source identifier, in other words, a brand name identifies a product and/or service as exclusively coming from a single commercial source (i.e., company). An example of a brand name is PAMPERS®, which is also a trademark. Absorbent articles of the present invention may be marketed and/or packaged under the same brand name. In addition to the brand name, a product descriptor (e.g., Extra Absorbent) or other insignia (e.g., Swaddlers® may also be associated with the absorbent article.

“Outer cover” means a component of a wearable absorbent article that is adapted to be worn about the lower torso of a wearer, and is adapted to support or in normal use is capable of supporting and holding an absorbent insert. The term encompasses a wrapping structure (such as included in a conventional diaper) and a pant structure (such as included in underwear for the lower torso, of any design).

“Disposed” refers to an element being located in a particular place or position. A feature that is disposed on a surface or side of a component may be integral with said component or may be joined to said component.

“Disposable,” when referring to an absorbent insert, means that the absorbent insert is not adapted or intended to be effectively sanitarily laundered in an ordinary household laundering process and ordinary household equipment, and thereby is ordinarily unsuitable for sanitary and effective reuse so as to provide as-new intended functions and performance, following soiling by exudates and removal from an outer cover. By way of non-limiting examples, effective laundering may be frustrated or prevented, causing the insert to be disposable, by inclusion of materials and/or construction: that do not retain their substantial as-new physical shape or structure through ordinary household laundering and drying so as to be effective as-new in reuse; that absorb aqueous liquids and cannot be sufficiently dried/dehydrated in ordinary household drying equipment and ordinary drying cycles so as to be effective as-new in reuse; that dissolve or substantially degrade in ordinary household laundering or drying, causing the insert to be substantially damaged or rendered useless; and/or that cannot be effectively cleaned of exudate material through ordinary laundering, so as to be sanitary and otherwise acceptable for re-use.

“Insignia” as used herein means objects, character representations, words, colors, shapes or other indicia that can be used to distinguish, identify or represent the manufacturer, retailer, distributor and/or brand of a product, including but not limited to trademarks, logos, emblems, symbols, designs, figures, fonts, lettering, crests or similar identifying marks.

### Overview

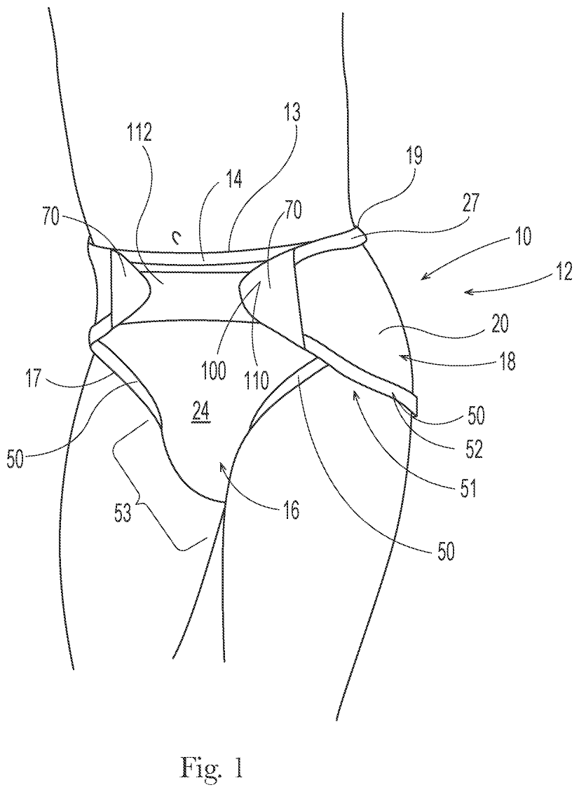

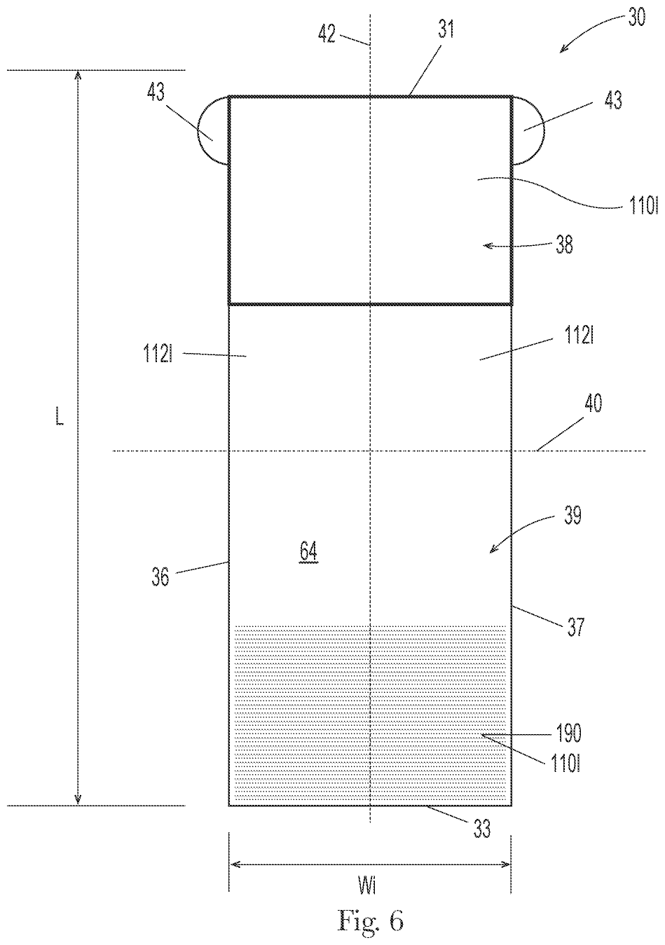

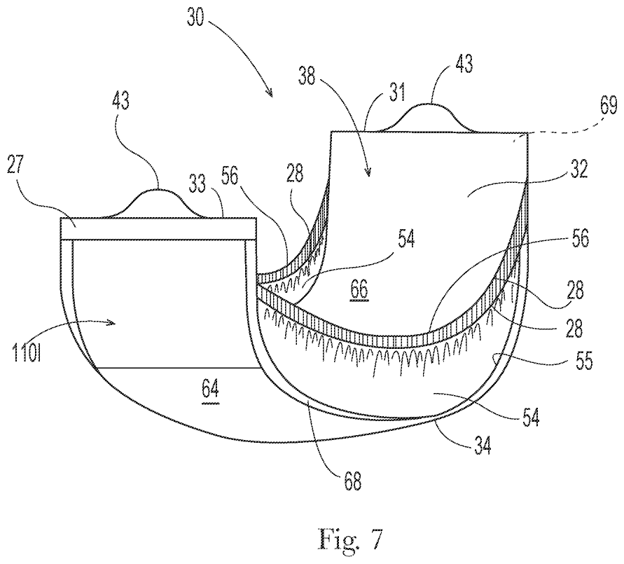

FIG. 1 is a perspective view an exemplary, nonlimiting embodiment of an absorbent article 10. The article is shown in the form of a two-piece absorbent article 12, having an outer cover 20 and an absorbent insert 30 shown in FIGS. 5-6. It is to be understood that during manufacturing, the article may comprise several discrete pieces that are joined together. However, by two-piece absorbent article, it is meant that the article in its final form has two components that the user assembles together for wear. While shown as a two-piece absorbent article, it is also contemplated that the article may comprise more than two pieces, such as attachable ears or belts. Returning to FIG. 1, it can be seen that the absorbent article 10 may have a front region 14, a rear region 18 and a crotch region 16 disposed between the front and rear regions. The article may be placed on a wearer by wrapping the outer cover 20 between the wearer's legs and under the buttocks such that the crotch region 16 is between the wearer's legs. When the insert 30 has been installed into outer cover 20, the insert 30 will then be disposed within outer cover 20, next to the wearer. Nonlimiting examples of two-piece articles are disclosed in U.S. Pat. Nos. 8,998,870, 9,089,456, 8,435,223, 9,011,402, 8,808,263, 8,759,605 and 8,932,273 and 9,078,789.

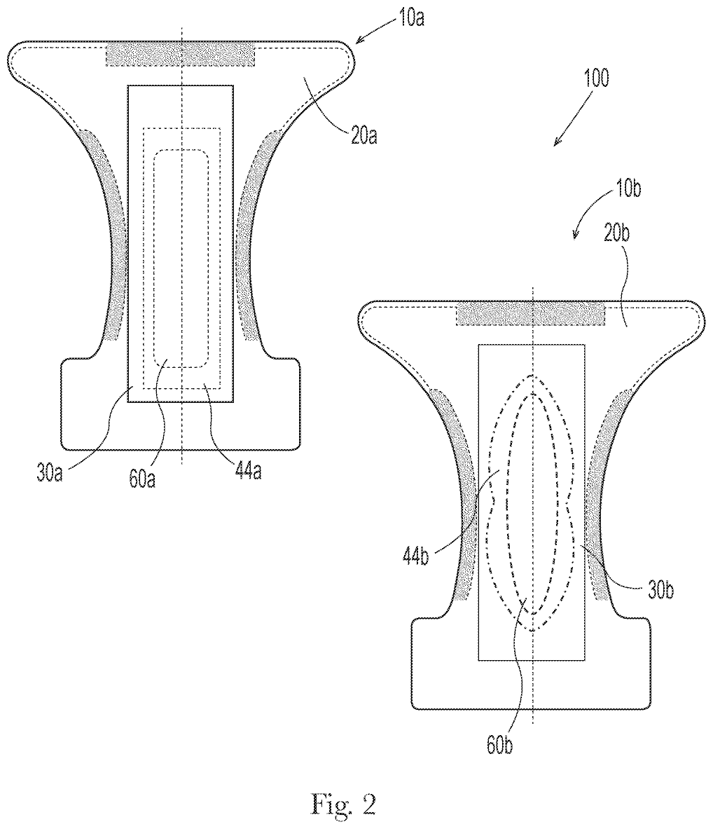

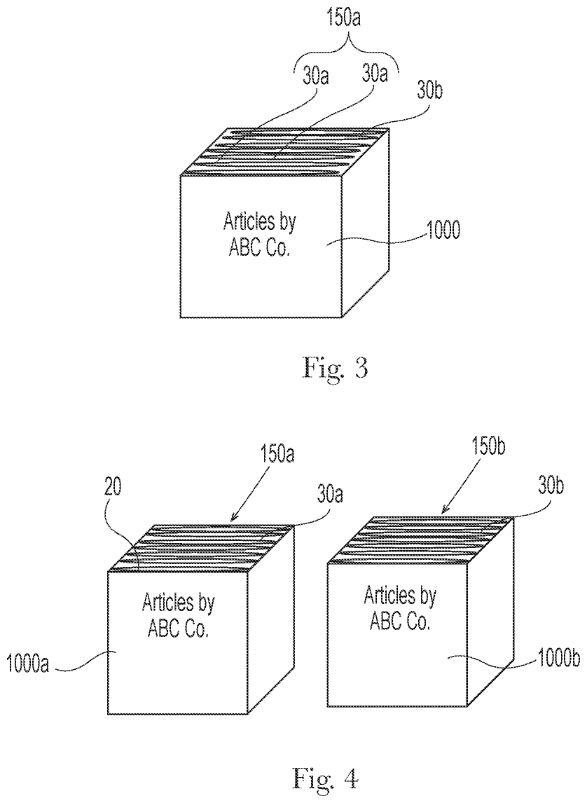

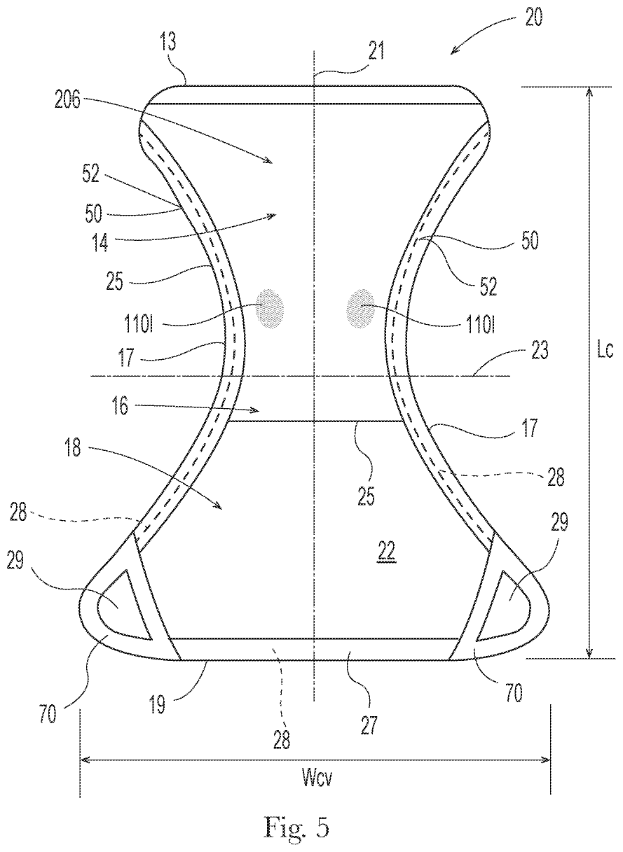

Articles may be provided in an array 100 as shown in FIG. 2. The array 100 comprises at least a first absorbent insert 30a and a second absorbent insert 30b. The first and second absorbent inserts differ in mass by 15% or less while differing in absorbent capacity by at least 15%. The two inserts may be provided in different packages 1000a, 1000b in various embodiments as shown in FIG. 4. Alternatively, the two inserts may be provided in the same package 1000 (FIG. 3). The array may further include one or more reusable outer covers 20. These and additional features are discussed in more detail below.

Two-Piece Article As shown in FIGS. 5-6, an absorbent article in accordance with present disclosure comprises an outer cover and an absorbent insert. In various embodiments, the absorbent insert is disposable and the outer cover is reusable. Turning to FIG. 5, an exemplary outer cover 20 is shown in a flat configuration with the wearer-facing surface 22 facing the viewer. The outer cover comprises a front lateral edge 13, a rear lateral edge 19, and two longitudinal edges 17. The outer cover comprises a length, Lc, from the outboard most portion of the front lateral edge to the outboard-most portion of the rear lateral edge. The outer cover may comprise a lateral axis 23, dividing evenly its length, and a longitudinal axis 21. The longitudinal edges 17 may be parallel to the longitudinal axis 21. However, for better fit, longitudinal edges 17 may be curved or angled to produce, for example, an “hourglass” shape article when viewed in a plan view as shown in FIG. 2, for example. In nonlimiting examples, the outer cover may be asymmetric, for instance having a width its rear region that is greater than the width in its front region. Thus, the width of the outer cover, Wcv, as measured between the longitudinal edges along a line parallel to the lateral axis, may vary along the longitudinal length of the outer cover. Nonlimiting examples of outer covers are disclosed in U.S. Pat. Nos. 9,387,138 and 8,435,223.

Outer cover 20 and/or layers or portions thereof may be made of any durable or semi-durable knitted, woven or nonwoven textile or textile-like material that is appropriately compatible with skin of the intended wearer(s). Suitable examples are described in U.S. application Ser. Nos. 12/687,493; 12/687,412; 12/687,528; and Ser. No. 12/687,425 (all by Roe et al.).

Non-limiting examples of fibers, nonwovens and laminates of nonwovens and films that might be considered for use as semi-durable outer cover materials may be found in U.S. Pat. Nos. 7,223,818; 7,211,531; 7,060,149; 6,964,720; 6,905,987; 6,890,872; 6,884,494; 6,878,647; and 5,518,801; and U.S. Published Applications Nos. 2008/0319407; 2008/0045917; 2007/0293111; 2007/0287983; 2007/0287348; 2007/0249254; 2007/0203301; and 2005/0164587.

The outer cover 20 may be formed of a single layer of a durable or semi-durable material or may be formed from two or more layers, which may be joined together at one or more seams 25. In nonlimiting examples, a garment-facing surface 24 is formed form a different material than a wearer-facing surface. For example, materials forming the garment-facing surface of the outer cover may comprise greater hydrophobicity than materials forming the wearer-facing surface. Likewise, different regions of the same surface may be formed by different materials. For example, the material predominately forming the inner surface of rear region 18 may be selected primarily for its elasticity features, which may better serve to provide snug fit about wearer body contours and accommodate wearer movement (i.e., about the buttocks and hips). By comparison, the material predominately forming the inner surface of front region 14 and/or crotch region 16 might be selected primarily for its hydrophobicity or hydrophilicity, which may better serve to contain liquid exudates.

Additionally, in some circumstances, it may be desirable that the material(s) selected for inner surfaces 22 have soft tactile properties so as to have a pleasant feel against the skin, particularly in areas where no portion of an insert is expected to be present between the outer cover and the wearer's skin. Further, it may be desirable that at least a portion of the inner surface comprise a material that is engageable by fastening components. Additionally, or alternatively, a second layer of material may be formed of a textile material having enhanced elasticity, such as by inclusion of fibers of an elastomeric material (such as spandex). In another example, an intermediate film layer may be included, laminated or not laminated with another layer.

Layers or other elements of the outer cover may be joined to each other via any suitable mechanism, including, for example, adhesives, mechanical bonding, ultrasonic bonding, sewing, stitching, serging, edging, and the like.

Waist Features, Leg Gasketing Systems, Ears

Referring again to FIGS. 1 and 5, the article and outer cover may comprise one or more waist features 27. The waist feature may be elastic and thereby provide better fit about the waist of the wearer. Elasticized waist features include waistbands, waist cuffs having pockets formed from a portion of the waist feature that is unattached from the remainder of the outer cover, and waist panels and/or belts designed to fit securely about the abdomen of the wearer. Nonlimiting examples of elasticized waist features are disclosed in U.S. patent application Ser. Nos. 14/533,472; 15/074,675 and 62/855,001. Elasticized waist features may comprise one or more nonwoven or textile layers, which may be layers of the outer cover or discrete portions, and one or more elastic elements 28. In nonlimiting examples, the elasticized waist feature comprises elastic strands joined to the nonwoven and/or textile layer(s). In further nonlimiting examples, the elasticized waist feature comprises a laminate of one or more textile and/or nonwoven layers and one or more films. The elasticized waist element may comprise one or more rugosities if the elastic material is strained prior to lamination. In other nonlimiting examples, the layers of the elastic laminate may be joined at zero applied strain and subsequently activated.

In alternative embodiments, the waist feature may be inelastic. In such configurations, the waist feature may provide additional anchoring about the waist of the wearer.

Additionally, or alternatively, the article may comprise a leg gasketing system 50, portions of which may be formed by the outer cover and/or the absorbent insert. The outer cover may include one or more elastic elements 28, such as films or elastic strands, extending through all or a portion of the leg opening 51 to form a leg band portion 52. The elastic elements may be laminated with one or more nonwoven layers and/or one or more textile layers. As described with respect to the waist feature, the leg band portion may include rugosities as result of the lamination process. In other nonlimiting examples, the leg band portion may be a zero strain, activated laminate.

The waist features and/or leg band portions may be disposed along the edge of the outer cover, and in some circumstances, it may be desired to have elasticized waist and leg band portions situated along substantially the entire length of the leg and/or waist openings so as substantially or completely encircle the wearer's legs and/or waist while outer cover 20 is worn. The gathered material within rugosities can serve to accommodate stretching of waist feature and leg band portions. This arrangement not only may provide for better fit about the wearer's legs, but also may enable the outer cover 20, when formed of appropriately sized and shaped material, to form a pouch-like structure 53 in the crotch region (see FIG. 1) when worn, which may serve to provide space within the outer cover to accommodate the insert 30 and help hold it in place within outer cover 20, in a substantially laterally centered position within the crotch region. The outer cover 20 may also include anchoring supplements, bands or systems thereof as described in more detail in U.S. Pat. No. 8,932,273.

The outer cover may include ears 70 in one or both of the front and back regions. As shown in FIGS. 1 and 5, the ears may include fastening components 110 such that the ears can be secured to the opposing region (e.g., rear ears may include fastening components that may engage with the front region). The outer cover may include receiving components 112 to operatively engage with fastening components 110. Nonlimiting examples of engageable fastening and receiving components include tape tabs, hook and loop fastening components, interlocking fasteners such as tabs & slots, buckles, buttons, snaps, and/or hermaphroditic fastening components. Some exemplary surface fastening systems are disclosed in U.S. Pat. Nos. 3,848,594; 4,662,875; 4,846,815; 4,894,060; 4,946,527; 5,151,092; and 5,221,274. An exemplary interlocking fastening system is disclosed in U.S. Pat. No. 6,432,098. A fastening component may be discrete from and joined to the outer cover or may be integral with the outer cover. The receiving component may be discrete from and joined to the outer cover or may be integral with the outer cover. The fastening component and/or the receiving component may further include a release tape or other material, including folded material, that protects the component from insult prior to use. In nonlimiting examples, a fastening component 110 is disposed on a wearer-facing surface 22 of the outer cover and a receiving component 112 is disposed on a garment-facing surface 24. In such nonlimiting examples, the outer cover may comprise two or more fastening systems having distinct fastening locations, which deconcentrates lateral tensile focuses and reduces the tendency of the front portion of the article to pivot around the single fastening location. Suitable dual fastening systems are disclosed for example in U.S. patent application Ser. Nos. 16/684,230 and 16/545,425.

The outer cover may also comprise one or more insert fastening components 110I capable of operatively engaging with an insert receiving component 1121 disposed on the absorbent insert, as shown in FIGS. 5-6. The insert fastening and receiving components may comprise any of the exemplary components disclosed.

The fastening and/or receiving components may be discrete from and joined to the outer cover or absorbent insert or may be integral with one or both. In nonlimiting examples, fibrous material (such as nonwoven material forming portions of the outer cover surfaces or portions of the insert backsheet or topsheet) may be comprise integral loops material as illustrated in FIG. 6. Further to the above, it is to be understood fastening systems need not necessarily include respective components of a two-component fastening system. Rather, a fastening system may require only one component. By way of nonlimiting example, a fastener component on outer cover 20 may include a patch of adhesive; a structure having a region of relatively high coefficient of friction; a pocket 29; flap; strap; or other capturing, holding and/or retaining surface, device or structure. Thus, a receiving component is unnecessary. Referring to FIG. 5, in some nonlimiting examples, the outer cover 20 may include one or more pocket structures 29 situated on or along the inner surface 22. A pocket structure may be adapted to receive, fit and capture, for example, the forward edge and a portion of forward region 38 of insert 30.

Absorbent Insert

Returning to FIG. 6, the absorbent insert 30 may be designed to contain and/or absorb body exudates, and may be made of pliable materials as will be described further below. The insert 30 includes a forward region 38 and a rearward region 39, a first lateral end 31 and a second lateral end 33, a first longitudinal edge 36, a second longitudinal edge 37, a lateral axis 40 and a longitudinal axis 42. The insert 30 comprises a length L from the outboard-most portion of front edge 31 to the outboard-most portion of the rear edge 33. The lateral axis equally divides the length L. In nonlimiting examples, the insert length L is less than the length of the outer cover Lc.

The insert may comprise one or more insert fastening components 110I, such as one or more areas of the adhesive 190. The insert fastening components may be disposed proximate to one or more lateral ends of the absorbent insert. Additionally, or alternatively, the insert may comprise one or more insert receiving components 1121.

The longitudinal edges 36, 37 may be generally parallel to the longitudinal axis. Alternatively, the longitudinal edges 36, 37 may be curved, such as in an hour-glass configuration. Thus, the width of the insert, Wi, may vary. In nonlimiting examples, the width of the insert, Wi, is less than the width of the outer cover, Wcv, at one or more longitudinal positions. The width of the insert, Wi, may be less than the width of the outer cover, Wcv, throughout the length of the insert.

In various embodiments, the absorbent insert may comprise a surface area of at least about 400 cm², or at least about 450 cm², or at least about 475 cm², or from about 300 cm² to about 600 cm², or from about 400 cm² to about 500 cm², reciting for each range every 10 cm² increment therein, on its wearer-facing surface and/or on its garment-facing surface. In this way, the absorbent insert may accommodate relatively large wearers (toddlers) while accommodating smaller sized wearers (infants). In some embodiments, the absorbent insert may be foldable, further allowing the size to be adjusted for different wearers, as is disclosed in commonly assigned U.S. Pat. App. No. 63/028,021. The absorbent insert may comprise a caliper of about 20 mm or less, or about 15 mm or less, or from about 15 mm to about 30 mm, according to the Caliper Test Method herein.

Additionally, or alternatively, the absorbent insert 30 may comprise a Rewet of about 0.15 g or less, or about 0.1 g or less, or about 0.08 g or less, or about 0.07 g or less, or from about 0.4 g to about 0.1 g, or from about 0.05 g to about 0.08 g, reciting for each range every 0.001 g increment therein, according to the Rewet Test Method herein. Such low rewet values indicate fluids are less likely resurface to or through the topsheet, making the wearer feel wet and/or causing leakage.

Table 1 shows comparative data between known products and an embodiment in accordance with the present disclosure. Comparative Example 1 is a disposable absorbent insert manufactured by CHARLIE BANANA under product name CHARLIE BANANA Disposable Insert, available in the United States in May 2020. Comparative Example 2 is a disposable absorbent insert manufactured by GROVIA under product name GROVIA BioSoaker, available in the United States in May 2020. Inventive Example 1 is a disposable insert comprising 8 g of absorbent polymer material having a CRC of 27 g/g and an acquisition distribution system (as described in more detail below) comprising 4 g of cross-linked cellulosic materials. Inventive Example 2 is a disposable insert comprising 9 g of absorbent polymer material having a CRC of 27 g/g and an acquisition distribution system (as described in more detail below) comprising 4 g of cross-linked cellulosic materials. Inventive Example 3 is a disposable insert comprising 10.7 g of absorbent polymer material having a CRC of 27 g/g and an acquisition distribution system (as described in more detail below) comprising 5 g of cross-linked cellulosic materials. The samples were tested according to the Rewet Test Method herein.

TABLE 1

| Sample | Rewet (g) |
|---|---|
| Comparative Example 1 | 0.19 |
| Comparative Example 2 | 0.19 |
| Inventive Example 1 | 0.055 |
| Inventive Example 2 | 0.046 |
| Inventive Example 3 | 0.037 |

The insert also may include one or more grasp structures 43, such as side grasp structures extending from a longitudinal side or grasp structures extending from a lateral end 31, 33. The grasp structures may be provided to enable the user to quickly and easily grasp the insert, handle the insert during application and/or properly place the insert. In certain embodiments, the grasp structure may have a different tactile feel than surrounding or adjacent areas to distinguish the area and ease the user's identification of the grasp structures.

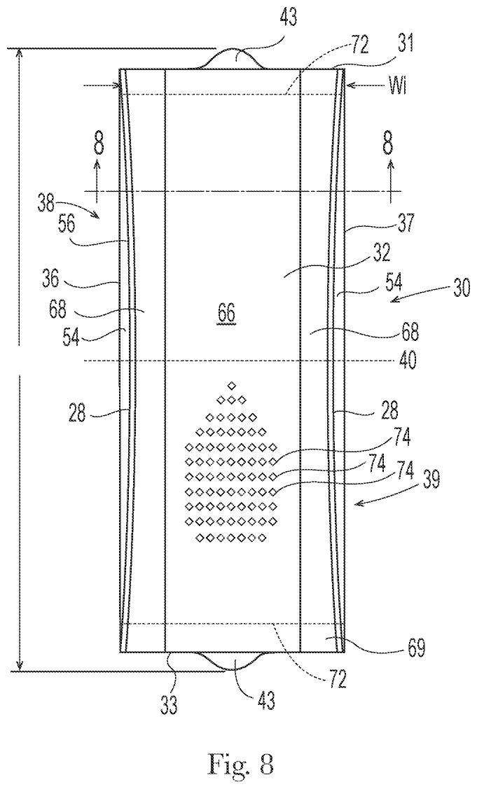

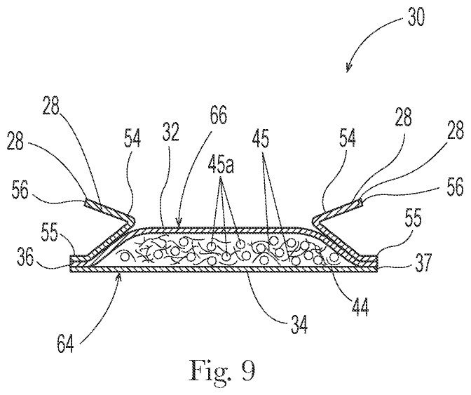

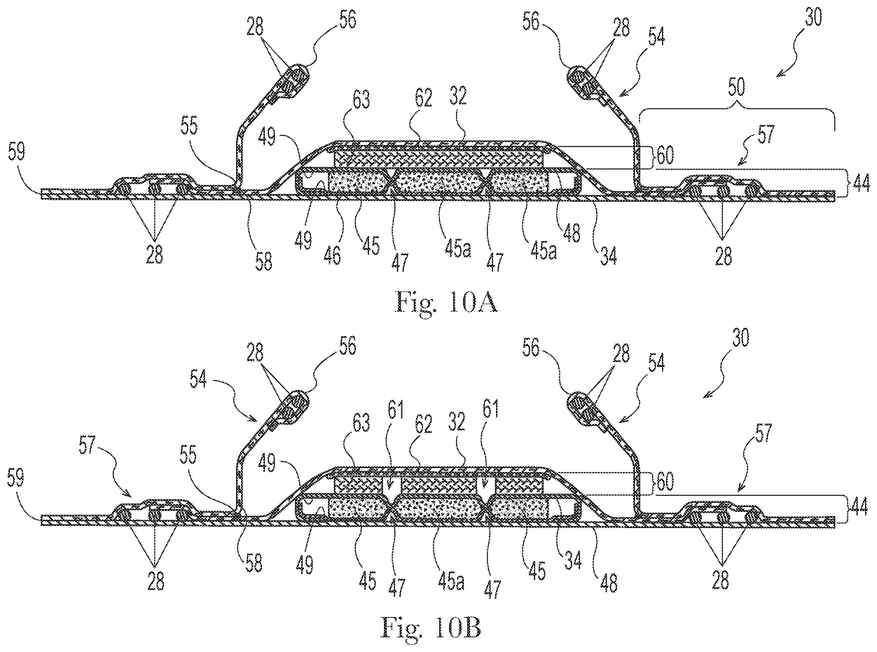

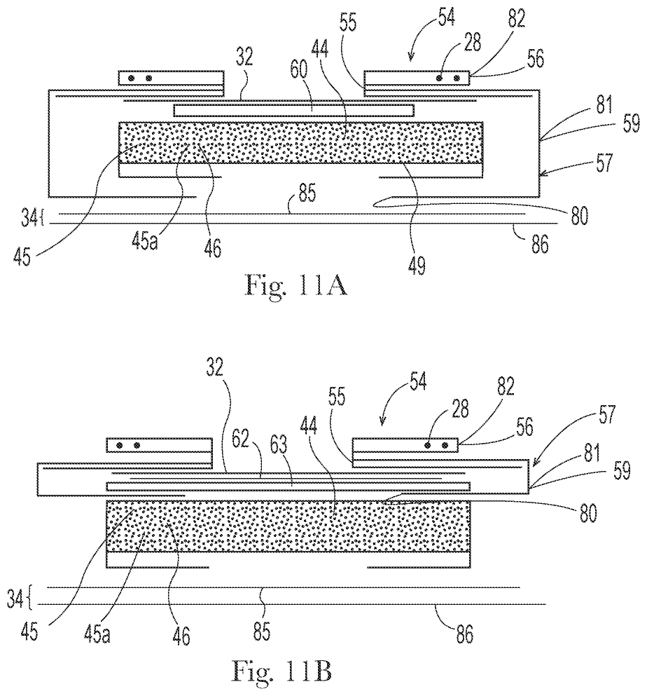

FIG. 7 depicts a disposable absorbent insert 30 in perspective view as it might appear in a free-standing, relaxed state, with both the body-facing surfaces 66 and garment-facing surfaces 64 shown. FIG. 8 depicts an example of an insert 30 shown stretched out and laid flat (against elastic-induced contraction to a position similar to that shown in FIG. 7), body-facing surfaces 66 facing the viewer. FIG. 9 depicts an exemplary cross-section of FIG. 8 according to certain nonlimiting examples. FIGS. 10A-10B depict lateral cross sectional views of examples of inserts having a dual leg gasketing system, taken through a lateral axis of the insert example.

As shown in FIG. 7 for example, the insert 30 may have a topsheet 32 and a backsheet 34. The topsheet and backsheet may be joined together along longitudinal seams 68 and along lateral seams 69. An absorbent core 44 may be disposed between the topsheet and the backsheet as shown for example in FIG. 9.

Returning to FIGS. 7-8, the insert 30 may further include longitudinal standing cuffs 54 affixed along the longitudinal sides 36, 37. Nonlimiting examples of absorbent inserts and details of their features are disclosed in U.S. Pat. Nos. 8,546,641 and 9,011,402. Stiffening elements 72 may also be included to aid the user in engaging the insert with the outer cover and/or help the insert maintain its intended shape and configuration during wear.

It will be appreciated that the outer cover described above can be constructed of materials and construction so as to bear and sustain a majority of the structural loading generally imposed upon a disposable diaper, by stretching and accommodation of the wearer's anatomical features and body movements, and by absorption, swelling and added weight resulting from the wearer's exudations of waste. Thus, lesser requirements for structural strength of an insert might be present with use of such an outer cover, as compared with strength required of inside components of a disposable diaper. Therefore, an article such as described herein may include a disposable absorbent insert manufactured from materials that are different from those ordinarily used in the manufacture of disposable diapers, such as petroleum-derived materials, e.g., polyethylene and polypropylene. For example, a disposable absorbent insert having one or more of a topsheet, backsheet, standing cuffs and/or other components formed of products of wood, cotton, flax (linen), hemp, bamboo, or other cellulose fibers (e.g., paper), in addition to the materials identified above, is contemplated. If resistance to aqueous liquid penetration or substantial liquid impermeability is desired, e.g., for a backsheet and/or standing cuffs, a material formed of ordinarily hydrophilic fibers such as paper may be coated or impregnated with a hydrophobic material, such as a skin-compatible oil or wax, to impart the desired resistance to aqueous liquid penetration. Each of the materials forming the insert may be selected so as to be dispersible in water or an aqueous solution, flushable, biodegradable and/or compostable (preferably to an agriculturally usable humus or soil amendment).

Topsheet

The topsheet 32 is generally a portion of the absorbent article 10 that may be positioned at least in partial contact or close proximity to a wearer. Suitable topsheets 32 are generally supple, soft feeling, and non-irritating to a wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured, may have any suitable three-dimensional features, and/or may have a plurality of embossments (e.g., a bond pattern). The topsheet 32 may comprise one or more apertures 74. The topsheet may be apertured by overbonding a material and then rupturing the overbonds through ring rolling, such as disclosed in U.S. Pat. No. 5,628,097, to Benson et al., issued on May 13, 1997 and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

Topsheet 32, backsheet 34 or any portion of the top sheet or backsheet may be embossed and/or matte finished to provide a more cloth-like appearance.

Backsheet

Backsheet 34 is generally the outer liner portion of insert 30 forming the garment-facing surface 64 thereof, and prevents the exudates absorbed and contained within insert 30 from wicking through and soiling the outer cover.

The backsheet 34 may comprise one or more nonwovens, films, elastomeric films, foams, strands, or combinations of these or other suitable materials with nonwovens or synthetic films. In nonlimiting examples, the backsheet is a laminate of a film 85, and a nonwoven 86.

In certain embodiments, the backsheet 34 is substantially water-impermeable. The backsheet may, for example, be or comprise a thin plastic film, such as a thermoplastic film having a thickness of about 0.012 mm to about 0.051 mm. Other suitable backsheet 34 materials may include breathable materials that permit vapors to escape from the absorbent article 10 while still preventing exudates from passing through the backsheet 34.

In certain embodiments, the backsheet 34 may have a water vapor transmission rate (WVTR) of greater than about 2000 g/24 h/m², greater than about 3000 g/24 h/m², greater than about 5000 g/24 h/m², greater than about 6000 g/24 h/m², greater than about 7000 g/24 h/m², greater than about 8000 g/24 h/m², greater than about 9000 g/24 h/m², greater than about 10000 g/24 h/m², greater than about 11000 g/24 h/m², greater than about 12000 g/24 h/m², greater than about 15000 g/24 h/m², measured according to WSP 70.5 (08) at 37.8° C. and 60% Relative Humidity. A higher WVTR may be desired in this particular application, since the insert backsheet 34 will not form the outer surface of the wearable article, as a conventional disposable diaper backsheet would, but rather, will be covered by the one or more layers of the outer cover material(s)—which themselves may act in some circumstances to reduce WVTR of the composite structure.

Other suitable materials and/or manufacturing techniques may be used to provide a suitable backsheet 34 including, but not limited to, surface treatments, particular film selections and processing, particular filament selections and processing.

Backsheet 34 may be joined to topsheet 32, absorbent core 44 or any other element of insert 30 by any suitable attachment mechanism known in the art.

Absorbent Core

Turning to FIG. 9, the insert 30 may have an absorbent core 44 disposed within the envelope-like structure formed by the topsheet 32 and backsheet 34. The absorbent core 44 may comprise a wide variety of liquid-absorbent materials 45 commonly used in disposable diapers and other absorbent articles. Examples of suitable absorbent materials include comminuted wood pulp, which is generally referred to as air felt creped cellulose wadding; melt blown polymers, including co-form; chemically stiffened, modified or cross-linked cellulosic fibers; tissue, including tissue wraps and tissue laminates; absorbent foams; absorbent sponges; superabsorbent polymers; absorbent gelling materials (AGM); or any other known absorbent material or combinations of materials. The amount of absorbent material, such as absorbent particulate polymer material 45a present in the absorbent core may vary, but in certain embodiments, is present in the absorbent core in an amount greater than about 80%, or greater than about 85%, or greater than about 90%, or greater than about 95% by weight of the core. In nonlimiting examples, a thermoplastic material, such as a thermoplastic adhesive composition 46, may be used to immobilize superabsorbent particles on a substrate (e.g., the topsheet, backsheet or core wrap).

Absorbent particulate polymer material 45a used in the core may have a CRC (centrifuge retention capacity) value of more than 20 g/g, or more than 22 g/g, or more than 25 g/g, for example up to 50 g/g, or up to 40 g/g, or to 30 g/g, as measured according to EDANA method WSP 241.2-05. The CRC measures the liquid absorbed by the superabsorbent polymer particles for free swelling in excess liquid. Superabsorbent polymer particles having a high CRC value may be preferred since less superabsorbent polymer particles are needed to facilitate a required overall capacity for liquid absorption.

The absorbent insert may be provided with an absorbent capacity of at least about 150 g, or at least about 200 g, or at least about 250 g, or from about 120 to about 450 g, reciting for said range every 10 increment therein. The absorbent capacity is the mathematical product of the mass of absorbent material in the insert and the CRC of said material.

In certain embodiments, at least a portion of the absorbent core is substantially cellulose free and contains less than 10% by weight cellulosic fibers, less than 5% cellulosic fibers, less than 1% cellulosic fibers, no more than an immaterial amount of cellulosic fibers or no cellulosic fibers. It should be understood that an immaterial amount of cellulosic material does not materially affect at least one of the thinness, flexibility, and absorbency of the portion of the absorbent core that is substantially cellulose free. Among other benefits, it is believed that when at least a portion of the absorbent core is substantially cellulose free, this portion of the absorbent core is significantly thinner and more flexible than a similar absorbent core that includes more than 10% by weight of cellulosic fibers.

In some embodiments, as shown in FIG. 10A, the absorbent core may comprise one or more channels 47, wherein said channels are substantially free of absorbent particulate polymer material. The channels 47 may extend longitudinally or laterally. The absorbent core may further comprise two or more channels. The channels may be straight, curvilinear, angled or any workable combination thereof. In nonlimiting examples, two channels are symmetrically disposed about the longitudinal axis.

The absorbent core 44 may include a core wrap 48, comprising one or more substrates 49 to encloses the absorbent material 45. Where channels are present, the core wrap may be bonded within one or more channels, thereby providing permanent channels which maintain their channel structure in the wet state.

The absorbent core 44 may be manufactured in a wide variety of sizes and shapes (e.g., rectangular, hourglass, “T”-shaped, etc.). The configuration and construction of absorbent core 44 may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures). Exemplary absorbent structures for use as the absorbent core 44 are described in U.S. Pat. Nos. 7,744,576; 9,072,634 and U.S. patent application Ser. Nos. 13/491,642 and 15/232,901.

Acquisition-Distribution System

Referring to FIGS. 10A-10B, in some embodiments, an acquisition-distribution system (ADS) 60 is disposed between the topsheet 32 and the absorbent core 44. One function of the ADS is to quickly acquire one or more of the fluids and distribute them to the absorbent core in an efficient manner. The ADS may comprise one, two or more layers, which may form a unitary layer or may remain as discrete layers which may be attached to each other. The ADS 60 may include hydrophilic materials that provide significant wicking of bodily exudates. These materials may dewater the topsheet 32 and quickly move bodily exudates into the absorbent core 44. The ADS 60 may comprise one or more nonwoven materials, foams, cellulosic materials, cross-linked cellulosic materials, air laid cellulosic nonwoven materials, spunlace materials, or combinations thereof, for example. In some instances, portions of the ADS 60 may extend through portions of the topsheet 32, portions of the topsheet 32 may extend through portions of the ADS 60, and/or the topsheet 32 may be nested with the ADS 60. Typically, an ADS 60 may have a width and length that are smaller than the width and length of the topsheet 32. The ADS may have one or more channels 61 as described above with reference to the absorbent core 44. The channels 61 in the ADS may align or not align with channels 47 in the absorbent core 44. In an example, a first ADS layer may comprise an acquisition layer 62 suitable for quickly acquiring fluids, and the second layer 63 may comprise a distribution layer capable of distributing fluids. In nonlimiting examples, the acquisition layer 62 may comprise a nonwoven material and/or the distribution layer 63 may comprise a cross-linked cellulosic material. Suitable ADS are described in WO 2000/59430, WO 95/10996, U.S. Pat. No. 5,700,254, and WO 02/067809, for example.

Cuff Structures

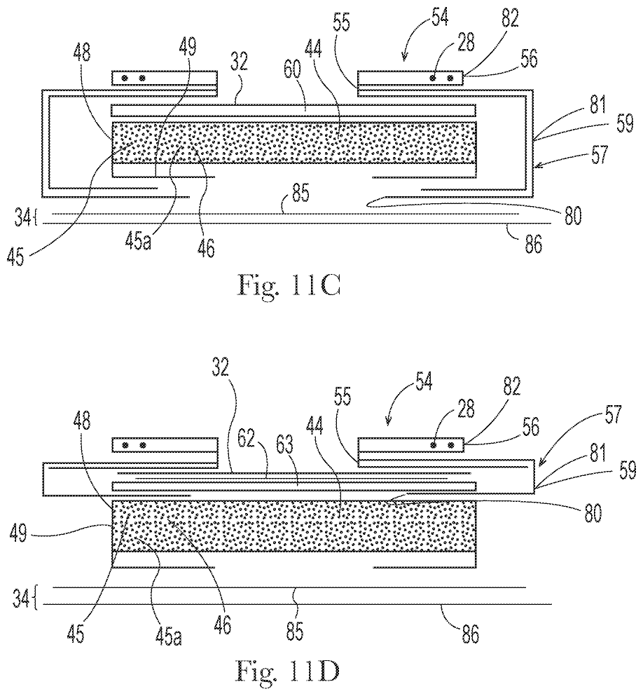

As noted above, the article 10 may comprise a leg gasketing system 50, portions of which may be formed by the insert 30. The leg gasketing system comprises one or more cuffs. At least one cuff comprises a material edge that may be disposed below the topsheet as shown in FIGS. 11A-11B. More particularly, the material edge may be disposed between layers, where each such layer is disposed beneath the topsheet (i.e., the material edge does not contact the topsheet). In nonlimiting examples, the at least one cuff comprises a laminate of two or more layers, and each may have a material edge disposed below the topsheet (i.e., material edge does not contact the topsheet) and discussed in more detail below.

Returning to FIGS. 7-8, the insert may comprise a pair of longitudinal standing cuffs 54, also referred to as barrier leg cuffs. Each standing leg cuff may be formed by a piece of material which is bonded to the absorbent insert so it may extend upwards from a wearer-facing surface and provide improved containment of fluids and other body exudates approximately at the junction of the torso and legs of the wearer. The standing leg cuffs are delimited by a proximal edge 55 joined directly or indirectly to the topsheet 32 and/or the backsheet 34 and a free terminal edge 56, which is intended to contact and form a seal with the wearer's skin. In some embodiments, the free terminal edge 56 comprises a folded edge. The standing leg cuffs 54 extend at least partially between the front edge 31 and the rear edge 33 on opposite sides of the longitudinal centerline 42 and are at least present in the crotch region.

The standing leg cuffs may be integral with the topsheet 32 or the backsheet 34 or may be a separate material joined to the topsheet and/or backsheet. Each standing leg cuff 54 may comprise one, two or more elastic elements 28 close to the free terminal edge 56 to provide a better seal. The standing cuff may be formed of any of a variety of substrates such as plastic films and woven or nonwoven webs of natural fibers (e.g., wood or cotton fibers), synthetic fibers (e.g., polyester or polypropylene fibers), or a combination of natural and synthetic fibers. In certain embodiments, the standing cuffs may be formed of a substantially liquid impermeable web to contain and isolate liquid exudates from the outer cover, outer clothing and environment of the wearer and/or may be formed of a vapor permeable web for breathability of the insert and article.

Referring to FIGS. 10A-10B, the article 10 may comprise a dual gasketing system, which includes the standing cuffs and gasketing cuffs 57. The gasketing cuffs 57 may be joined to the insert 30, more particularly to the topsheet and/or backsheet. The gasketing cuffs are disposed outboard of the standing cuffs and may provide a better seal around the thighs of the wearer. A gasketing cuff 57 may comprises a proximal edge 58 and a free terminal edge 59. The free terminal edge 59 may comprise a folded edge 81.

In one example shown in FIGS. 11A-11B, a gasketing cuff 57 may comprises a material edge 80 and a free terminal edge 59. The free terminal edge 59 may comprise the folded edge 81. The gasketing cuffs may be folded such that the material edge 80 is disposed below the topsheet. The material edge may be disposed between the absorbent core 44 and the backsheet 34 as shown in FIG. 11A, or more particularly between the backsheet film 85 and the absorbent core substrate 49. In this way, the gasketing cuff material may provide an additional barrier layer to the absorbent material while providing a finished, smooth edge for the wearer (i.e., folded edge 81). It is also contemplated that a gasketing cuff material edge may be disposed between the backsheet film 85 and the backsheet nonwoven 86. The gasketing cuff material edge 80 may be disposed between the acquisition distribution system 60 and the absorbent core 44, as shown in FIG. 11B. More particularly, the gasketing cuff material edge 80 may be disposed between a core substrate 49 and the acquisition distribution system 60, as shown in FIG. 11B. In other configurations, the gasketing cuff material edge 80 may be disposed between layers of the acquisition distribution system 60, for example between an acquisition layer 61 and a distribution layer 62. These latter configurations permit materials of the acquisition distribution system may be further enclosed while providing a finished, smooth edge for the wearer (i.e., folded edge 81).

Each gasketing cuff may comprise one or more elastic elements 28, which may be sandwiched between other layers of material, such as the portions of material forming the attached proximal portions of the standing cuffs, topsheet, backsheet, separate gasketing cuff material, or combinations thereof. The gasketing cuff may be formed of any of a variety of substrates such as plastic films and woven or nonwoven webs of natural fibers (e.g., wood or cotton fibers), synthetic fibers (e.g., polyester or polypropylene fibers), or a combination of natural and synthetic fibers. As noted with respect to standing cuffs 54, gasketing cuffs may likewise be formed of any suitable web materials but preferably are formed of web materials that are effectively liquid impermeable while being vapor permeable, so as to contain the wearer's liquid exudates within the insert while permitting the insert to “breathe” to avoid excess humidity within the insert (which may overhydrate the wearer's skin and promote conditions such as diaper rash). Suitable materials include nonwoven, films, elastic strands and combinations thereof.

In further embodiments, the leg gasketing system comprises standing leg cuffs that are integral with gasketing cuffs. Some suitable leg gasketing systems which may be part of the absorbent article are disclosed in U.S. Pat. App. No. 62/134,622, 14/077,708; U.S. Pat. Nos. 8,939,957; 3,860,003; 7,435,243; 8,062,279.

Masking Layers

One or more masking layers or materials may be provided in the absorbent articles 10, particularly in the absorbent insert. A masking layer may be a layer that provides a cushiony feel when the absorbent article is touched from the garment-facing surface or the wearer-facing surface. The masking layer may “mask” a grainy feel potentially caused by the absorbent material 45, such as superabsorbent polymers 45a. The masking layer may “mask” bodily exudates from being visible when viewing the wearer-facing surface or the garment-facing surface of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the backsheet or external layer of the outer cover material. The masking layer may be the layer forming the garment-facing side or the wearer-facing side of the absorbent core 44. The masking layer may be a separate material positioned intermediate the garment-facing side of the core 44 and the liquid impermeable backsheet 34.

Bio-Sourced Materials

Components of the disposable absorbent article can at least partially be comprised of bio-sourced content as described in U.S. Pat. Pub. Nos. 2007/0219521A1, 2011/0139658A1, 2011/0139657A1, 2011/0152812A1, and 2011/0139659A1. These components include, but are not limited to, topsheets, backsheet films, backsheet nonwovens, side panels, leg gasketing systems, superabsorbent, acquisition layers, core wrap materials, adhesives, outer covers, fastener systems, and landing zones. In at least one embodiment, a disposable absorbent article component comprises a bio-based content value from about 10% to about 100%, or from about 25% to about 75%, or from about 50% to about 60% using ASTM D6866-10, method B. In order to apply the methodology of ASTM D6866-10 to determine the bio-based content of any component, a representative sample of the component must be obtained for testing. In at least one embodiment, the disposable absorbent article component can be ground into particulates less than about 20 mesh using known grinding methods (e.g., WILEY® mill), and a representative sample of suitable mass taken from the randomly mixed particles.

### Arrays

Returning to FIG. 2, the present invention includes an array 100. Articles and article components in the array 100 may be manufactured and/or distributed by a single manufacturer, under a common brand name and/or under a common tradename or trademark. The array 100 includes a first absorbent insert 30a and a second absorbent insert 30b. The first and second absorbent inserts may comprise any of the features discussed above with respect to absorbent inserts. The absorbent inserts may comprise the same surface area or different surface areas. Likewise, the absorbent inserts may comprise the same caliper or different calipers, though both may be 20 mm or less in various embodiments.

The first absorbent insert comprises a first absorbent capacity. The first absorbent capacity may be at least about 150 g, or at least about 200 g, or at least about 250 g, or from about 120 g to about 300 g, reciting for said range every 10 g increment therein. The absorbent capacity is the mathematical product of the mass of absorbent material in the insert and the CRC of said material. The second absorbent comprises a second absorbent capacity. The second absorbent capacity is at least about 10%, or at least about 15%, or at least about 20%, or at least about 25% greater, or at least about 30% greater, or from about 5% to about 45% greater than the first capacity, or from about 10% to about 40%, reciting for each range every 1% increment therein. The second absorbent capacity may be at least 175 g, or least 200 g, or at least 300 g, or from about 175 g to about 500 g, or from about 250 g to about 475 g, or from about 300 g to about 450 g, reciting for each range every 10 g increment therein.

The first absorbent insert further comprises a first mass as determined by the Mass Test Method herein. The first mass may comprise about 25 g or less, or about 22 g or less, or about 20 g or less, or from about 15 g to about 25 g, or from about 20 g to about 22 g, reciting for said ranges each 0.5 g increment therein. The second absorbent insert comprises a second mass. The second mass is no more than about 26%, or no more than about 20%, or no more than about 15%, no more than about 10%, or from about 5% to about 28%, or from about 10% to about 25% greater than the first mass, reciting for each range every 1% increment therein. The second mass may comprise about 30 g or less, or about 28 g or less, or about 25 g or less, or from about 20 g to about 30 g, or from about 22 g to about 28 g, reciting for each range every 0.5 g increment therein.

In various embodiments, the absorbent capacity differs to a greater extent than the mass between the two articles. For example, the second absorbent capacity may be at least 20% greater, at least 25% greater, at least 30% greater, at least 35% greater, at least 40% greater, or at least 45% greater than the first absorbent capacity, while the second mass is less than 20% greater, less than 18% greater, less than 15% greater, or less than 12.5% greater than the first mass. In nonlimiting examples, the capacity increase is at least 1.1, at least 1.3, at least 1.5, at least 1.75, or at least 1.9 the mass increase, as calculated by dividing the difference in absorbent capacity by the difference in the mass.

In nonlimiting examples, the first absorbent insert comprises a first absorbent core 44a that is substantially cellulose free. Additionally, or alternatively, the second absorbent insert 44b comprises a second absorbent core that is substantially cellulose free. It is also contemplated that the first absorbent core and second absorbent differ by: type and/or combination of absorbent materials, weight of absorbent materials, type and/or basis weight of core wrap materials, absorbent capacity, caliper, size, shape and combinations thereof.

The first absorbent insert and the second absorbent insert may comprise acquisition distribution systems as described above. The first absorbent insert may comprise a first acquisition distribution system 60a, and the second absorbent insert may comprise a second acquisition distribution system 60b. The first acquisition distribution system may differ from the second acquisition distribution system by: types of materials forming the ADS, relative amounts of materials forming the ADS, basis weight, number of layers, arrangement of layers, mass, relative mass of particular layer(s), basis weight of particular layer(s), and combinations thereof. In nonlimiting examples, the mass of the first acquisition distribution system is at least about 10%, or at least about 15%, or least about 20%, or from about 10% to about 60% less than the weight of the second acquisition distribution system. It is also contemplated, however, that the first and second acquisition distribution systems comprise substantially the same weight.

The first and second absorbent inserts may comprise a Rewet of about 0.15 g or less according to the Rewet Test Method herein. In nonlimiting examples, the second absorbent insert comprises a Rewet value that is less than the first absorbent insert. For example, the first absorbent insert may comprise a Rewet value that is at least about 20% greater, or at least about 25%, or at least about 30% greater, or from about 10% to about 40%, than the Rewet value of the second absorbent insert, reciting for said range every 5% increment therein.

Each of the first and second absorbent inserts may comprise at least 10%, or at least 20%, or from about 20% to about 60% bio-based content. The first absorbent insert and second absorbent insert may comprise substantially the same amount of bio-based content, or may comprise different amounts.

The array 100 may further comprise one or more outer covers 20, having any of the features described above with respect to outer covers. An outer cover may be reusable and configured to join with the first absorbent insert and/or the second absorbent insert to form an absorbent article 10. As shown in FIG. 2, the array may comprise a first outer cover 20a and a second outer cover 20b. In nonlimiting examples, the first outer cover 20a may be configured to support the first absorbent insert 30a to form a first absorbent article 10a, and the second outer cover 20b may be configured to support the second absorbent insert 30b to form a second absorbent article 10b. Additionally, or alternatively, the first outer cover 20a may be configured to support the second absorbent insert to form an absorbent article and/or the second outer cover may be configured to support the first absorbent insert to form an absorbent article. The first outer cover may differ from the second outer cover by: size, graphics, color, material(s) from which is made and combinations thereof.

Turning to FIGS. 3-4, although described above as a single first absorbent insert and a single second absorbent insert, it is also contemplated that the array 100 may comprise a plurality 150a of first absorbent inserts, each having the features described above with reference to the first absorbent article. Additionally, or alternatively, the array 100 may comprise a plurality 150b of second absorbent inserts, each having the features described above with reference to the second absorbent article. Indicia may be used to indicate differences between the first and second absorbent insert. For example, indicia relating to night may be used for the second absorbent article, including for example, moons, stars, illustrations showing sleeping and the like. Additionally, or alternatively, indicia relating to daytime may be used for the first absorbent insert, including for example suns, illustrations showing waking and/or daytime activities, and the like.

The array may be disposed in one or more packages 1000. In the embodiment shown in FIG. 4, a first package 1000a comprises a first plurality 150a of first absorbent inserts 30a, and a second package 1000b comprises a second plurality 150b of second absorbent inserts 30b. At least one outer cover may be provided in the first package and/or in the second package. Additionally, or alternatively, an outer cover may be provided in a third package, separate from the first and second absorbent inserts. In the embodiment shown in FIG. 3, the first and second absorbent inserts may be provided in the same package 1000. At least one outer cover also may be provided in the package 1000. Additionally, or alternatively, an outer cover may be provided in a third package, separate from the first and second absorbent inserts.

The products in the array may be manufactured by the same manufacturer and/or under the same brand and/or trademark.

Example arrays of absorbent inserts are shown in Tables 2-4 below.

TABLE 2

Example Array 1

| | Absorbent | Absorbent |
|---|---|---|
| | Insert A | Insert B |
| Absorbent Gelling Material | 9.5 g | 11.5 g |
| Pulp | 4.0 g | 4.0 g |
| Nonwoven material(s) | 5.6 g | 5.6 g |
| Adhesives | 0.87 g | 0.87 g |
| Backsheet film | 0.79 g | 0.79 g |
| Release Material | 0.58 g | 0.58 g |
| Misc. (lotions, odor management | 0.25 g | 0.25 g |
| system, perfumes, etc.) | | |
| Elastics | 0.02 g | 0.02 g |
| Total Dry Weight (g) | 21.61 | 23.61 |
| Calculated Absorbent Capacity (g) | 261 | 316 |

Table 2 shows an example array having a first absorbent insert—Absorbent Insert A—and a second absorbent insert—Absorbent Insert B. Absorbent Insert B has approximately 2 g more absorbent gelling material as compared to Absorbent Insert A. The dry weight of Absorbent Insert B is 9.25% greater than the dry weight of Absorbent Insert B, however, the calculated absorbent capacity of Absorbent Insert B is approximately 21% greater than that of Absorbent Insert A.

TABLE 3

Example Array 2

| | Absorbent | Absorbent |
|---|---|---|
| | Insert B | Insert C |
| Absorbent Gelling Material | 11.5 g | 13.5 g |
| Pulp | 4.0 g | 4.0 g |
| Nonwoven material(s) | 5.6 g | 5.6 g |
| Adhesives | 0.87 g | 0.87 g |
| Backsheet film | 0.79 g | 0.79 g |
| Release Material | 0.58 g | 0.58 g |
| Misc. (lotions, odor management | 0.25 g | 0.25 g |
| system, perfumes, etc.) | | |
| Elastics | 0.02 g | 0.02 g |
| Total Dry Weight (g) | 23.61 | 25.61 |
| Calculated Absorbent Capacity (g) | 316 | 371 |

Table 3 shows an example array having a first absorbent insert—Absorbent Insert B—and a second absorbent insert—Absorbent Insert C. Absorbent Insert C has approximately 2 g more absorbent gelling material as compared to Absorbent Insert B. The dry weight of Absorbent Insert C is 8.5% greater than the dry weight of Absorbent Insert B, however, the calculated absorbent capacity of Absorbent Insert C is approximately 17% greater than that of Absorbent Insert B.

TABLE 4

Example Array 3

| | Absorbent | Absorbent |
|---|---|---|
| | Insert D | Insert C |
| Absorbent Gelling Material | 10.5 g | 13.5 g |
| Pulp | 4.0 g | 4.0 g |
| Nonwoven material(s) | 5.6 g | 5.6 g |
| Adhesives | 0.87 g | 0.87 g |
| Backsheet film | 0.79 g | 0.79 g |
| Release Material | 0.58 g | 0.58 g |
| Misc. (lotions, odor management | 0.25 g | 0.25 g |
| system, perfumes, etc.) | | |
| Elastics | 0.02 g | 0.02 g |
| Total Dry Weight (g) | 22.61 | 25.61 |
| Absorbent Capacity (g) | 289 | 371 |

Table 4 shows an example array having a first absorbent insert—Absorbent Insert D—and a second absorbent insert—Absorbent Insert C. Absorbent Insert C has approximately 3 g more absorbent gelling material as compared to Absorbent Insert D. The dry weight of Absorbent Insert C is 13.3% greater than the dry weight of Absorbent Insert D, however, the calculated absorbent capacity of Absorbent Insert C is approximately 28% greater than that of Absorbent Insert D.

### Combinations

A. An array (100) of absorbent articles (10) comprising:
   a first absorbent insert (30a) comprising a first absorbent capacity and a first dry mass; and
   a second absorbent insert (30b) comprising a second absorbent capacity and a second dry mass, wherein the second dry mass is no more than 15% greater than the first dry mass and the second absorbent capacity is at least 15% greater than the first absorbent capacity; and
   one or more reusable outer covers (20) configured to support the first absorbent insert (30a) to form a first absorbent article (10a) and/or configured to support the second absorbent insert (30b) to form a second absorbent article (10b).
B. The array according to paragraph A wherein the second absorbent capacity is at least 20% greater than the first absorbent capacity.
C. The array according to paragraphs A or B wherein the first dry mass is 25 g or less.
D. The array according to any of the preceding paragraphs wherein the second dry mass is 30 g or less.
E. The array according to any of the preceding paragraphs wherein the one or more reusable outer covers comprise a first reusable outer cover and a second reusable outer cover, wherein the first and second reusable outer cover differ by one of the group consisting of: graphics, color, size, material, and combinations thereof.
F. The array according to any of the preceding paragraphs wherein the first absorbent insert and the second absorbent insert each comprise a topsheet (32), a backsheet (34) and an absorbent core (44) disposed between the topsheet and the backsheet, and wherein at least one of the absorbent cores (44a, 44b) is substantially free of cellulosic material.
G. The array according to any of the preceding paragraphs wherein the first absorbent insert and second absorbent insert are disposable.
H. The array according to any of the preceding paragraphs wherein the first absorbent insert and second absorbent insert each comprise a caliper of 20 mm or less.
I. The array according to any of the preceding paragraphs wherein the first absorbent insert and the second absorbent insert each comprise standing cuff systems.
J. The array according to any of the preceding paragraphs wherein the first absorbent insert comprises a first acquisition distribution system (60a), and wherein the second absorbent insert comprises a second acquisition distribution system (60b).
K. The array according to paragraph J wherein the first and second acquisition distribution systems differ by one of the group consisting of: ADS materials, basis weight, layer configuration, mass, caliper and combinations thereof.
L. The array according to any of the preceding paragraphs wherein the first absorbent insert and second absorbent insert each comprise a rewet of 0.1 g or less.
M. The array according to any of the preceding paragraphs wherein the first absorbent insert and second absorbent insert each comprise a wearer surface area of at least 475 cm².
N. The array according to any of the preceding paragraphs wherein the first absorbent article and second absorbent article each comprise at least 10%, or at least 25%, bio-based content.
O. The array according to any of the preceding paragraphs wherein the first absorbent insert is disposed in a first package (1000a) and the second absorbent insert is disposed in a second package (1000b).
P. The array according to any of paragraphs A-N, wherein the first absorbent insert and the second absorbent insert are disposed in the same package (1000).
Q. The array according to any of the preceding paragraphs wherein each of the one or more reusable outer covers are configured to be used with the first absorbent insert and configured to be used the second absorbent insert.
R. The array according to any of the preceding paragraphs comprising a plurality of first absorbent inserts.
S. The array according to any of the preceding paragraphs comprising a plurality of second absorbent inserts.

### Caliper Test Method

The caliper, or thickness, of a test specimen is measured as the distance between a reference platform on which the specimen rests and a pressure foot that exerts a specified amount of pressure onto the specimen over a specified amount of time. All measurements are performed in a laboratory maintained at 23° C.±2 C.° and 50%±2% relative humidity and test specimens are conditioned in this environment for at least 2 hours prior to testing.

Caliper is measured with a manually-operated micrometer equipped with a pressure foot capable of exerting a steady pressure of 0.50 kPa±0.01 kPa onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm; however, a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Obtain a test specimen by removing it from an absorbent article, if necessary. When excising the test specimen from an absorbent article, use care to not impart any contamination or distortion to the test specimen layer during the process. The test specimen is obtained from an area free of folds or wrinkles, and it must be larger than the pressure foot.

To measure caliper, first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the caliper of the test specimen to the nearest 0.001 mm. In like fashion, repeat for a total of ten replicate test specimens. Calculate the arithmetic mean for all caliper measurements and report as Caliper to the nearest 0.001 mm.

### Mass Test Method

Mass is measured using an analytical balance (±0.01 g), for example Mettler Toledo XPS6002S, while the absorbent insert is dry and before use.

### Liquid Acquisition Time

Liquid Acquisition Time is measured for a test sample insulted with a known volume of test liquid, using a strikethrough plate and an electronic circuit interval timer described in compendial method WSP 70.3. The time required for the test liquid to pass into the test sample is recorded. Subsequent to the liquid acquisition test, a rewet test is performed on the test sample (described separately, herein). All measurements are performed in a laboratory maintained at 23° C.±2 C.° and 50%±2% relative humidity and test samples are conditioned in this environment for at least 2 hours prior to testing.

The test liquid used for this liquid acquisition test includes the following reagent grade components available from VWR International (or an equivalent source): Urea (NH₂CONH₂, CAS No 57-13-6), Sodium Chloride (NaCl, CAS No 7647-14-5), Magnesium Sulfate Heptahydrate (MgSO₄ 7H₂O, CAS No 10034-99-8), Calcium Chloride anhydrous (CaCl₂, CAS No 10043-52-4) and deionized water. To prepare the test liquid, add 2.0% w/v Urea, 0.90% w/v Sodium Chloride, 0.11% w/v Magnesium Sulfate Heptahydrate, 0.06% w/v Calcium Chloride anhydrous to a sufficiently sized volumetric flask. Then fill with deionized water (quantity sufficient) and mix thoroughly. The test liquid is used at room temperature.

The test apparatus includes a strikethrough plate, a baseplate, an electronic timer, a funnel with a magnetic valve to discharge the dose of test liquid and a ring stand to hold the funnel, in accordance with the apparatus descriptions described in compendial method WSP 70.3. A suitable apparatus is the Lister AC available from Lenzing Instruments GmbH & Co (Gampern, Austria). In addition, a micro pipette capable of delivering a 3.0 mL dose of test liquid is used.

Test samples are prepared by removing the absorbent article from any outer packaging, and if the article is folded, unfold it and extend the wings if present. The protective layer covering the panty fastening adhesive can be left in place. The test location is the intersection of the longitudinal and lateral midpoints of the absorbent article. Condition the test samples as previously described prior to testing.

Measure the liquid acquisition time as follows. The test sample is placed onto the baseplate, centering the test location over the plate. Place the strikethrough plate on top of the test sample with the test location centered under the center of the plate's orifice. Now place the entire stack of baseplate, test sample and strikethrough plate under the funnel, ensuring the test location is centered under the funnel. Adjust the height of the funnel so that it is 5±1 mm above the top surface of the strikethrough plate (i.e., 30 mm above the top surface of the test sample). Connect the electrodes of the strikethrough plate to the electronic timer and ensure the timer is set to zero. With the discharge valve of the funnel closed, use a micro pipette to dispense 3.0 mL of the test liquid into the funnel. Open the magnetic discharge valve of the funnel to discharge the 3.0 mL of test liquid into the reservoir of the strikethrough plate. The electronic timer will start as soon as the test liquid makes contact with the electrodes and will stop once the test liquid falls below the level of the electrodes (i.e., the entire liquid dose has penetrated into the test sample). Record the time indicated on the electronic timer to the nearest 0.01 seconds. The test sample is then immediately evaluated by one of the rewet tests described separately, herein (Standard Rewet or Instant Rewet). Prior to testing the next sample, the electrodes and strikethrough plate are cleaned with deionized water and dried completely.

In like fashion, the test sequence is repeated for five replicate test samples. Calculate and report Liquid Acquisition Time as the arithmetic mean of the replicates to the nearest 0.01 seconds.

### Standard Rewet

Standard Rewet is measured on a test sample 20 minutes after the Liquid Acquisition test (described separately, herein). Rewet is the amount of liquid that emerges from a previously wetted test sample when a weight is applied. All measurements are performed in a laboratory maintained at 23° C.±2 C.° and 50%±2% relative humidity.

The rewet weight is constructed of stainless steel, or equivalent, such that the dimensions of the bottom face of the weight are 4.5 cm by 10 cm and the total mass of the weight is 3150 g, including any handle that might be attached. A mechanical device can be constructed, if needed, to aid in the lowering and raising of the rewet weight.

For each test sample, two plies of filter paper cut to 150 mm diameter are used as the rewet substrate. The filter paper is conditioned at 23° C.±2 C.° and 50%±2% relative humidity for at least 2 hours prior to testing. A suitable filter paper has a basis weight of about 85 gsm, a thickness of about 180 microns with medium porosity, and is available from VWR International as Whatman grade 597.

Obtain the mass of 2 plies of the filter paper and record as Dry Mass_{fp} to the nearest 0.001 grams. When 20 minutes have elapsed after the Liquid Acquisition test, gently remove the strikethrough plate from the test sample and set aside. Place the 2 plies of pre-weighed filter paper onto the test sample, centering the stack over the dosing location. Now place the rewet weight centered over the top of the filter papers and start a 15 second timer. As soon as 15 seconds have elapsed, gently remove the rewet weight and set aside. Obtain the mass of the 2 plies of filter paper and record as Wet Mass_{fp} to the nearest 0.001 grams. Subtract the Dry Mass_{fp} from the Wet Mass_{fp} and report as Standard Rewet to the nearest 0.001 grams. Wipe off any residual test liquid from the bottom face of the rewet weight prior to testing the next sample.

In like fashion, repeat the entire procedure for five replicate samples. Calculate and report Standard Rewet as the arithmetic mean of the replicates to the nearest 0.001 grams.

### Instant Rewet

Instant Rewet is measured on a test sample 1 minute after the Liquid Acquisition test (described separately, herein). Rewet is the amount of liquid that emerges from a previously wetted test sample when a weight is applied. All measurements are performed in a laboratory maintained at 23° C.±2 C.° and 50%±2% relative humidity.

The rewet weight is constructed of stainless steel, or equivalent, such that the dimensions of the bottom face of the weight are 4.5 cm by 10 cm and the total mass of the weight is 3150 g, including any handle that might be attached. A mechanical device can be constructed, if needed, to aid in the lowering and raising of the rewet weight.

For each test sample, two plies of filter paper cut to 150 mm diameter are used as the rewet substrate. The filter paper is conditioned at 23° C.±2 C.° and 50%±2% relative humidity for at least 2 hours prior to testing. A suitable filter paper has a basis weight of about 85 gsm, a thickness of about 180 microns with medium porosity, and is available from VWR International as Whatman grade 597.

Obtain the mass of 2 plies of the filter paper and record as Dry Mass_{fp} to the nearest 0.001 grams. When 1 minute has elapsed after the Liquid Acquisition test, gently remove the strikethrough plate from the test sample and set aside. Place the 2 plies of pre-weighed filter paper onto the test sample, centering the stack over the dosing location. Now place the rewet weight centered over the top of the filter papers and start a 15 second timer. As soon as 15 seconds have elapsed, gently remove the rewet weight and set aside. Obtain the mass of the 2 plies of filter paper and record as Wet Mass_{fp} to the nearest 0.001 grams. Subtract the Dry Mass_{fp} from the Wet Mass_{fp} and report as Instant Rewet to the nearest 0.001 grams. Wipe off any residual test liquid from the bottom face of the rewet weight prior to testing the next sample.

In like fashion, repeat the entire procedure for five replicate samples. Calculate and report Instant Rewet as the arithmetic mean of the replicates to the nearest 0.001 grams.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of absorbent articles comprising:
a first package comprising a first plurality of first absorbent inserts, each of the first absorbent inserts comprising a first absorbent capacity; and
a second package comprising a second plurality of second absorbent inserts, each of the second absorbent inserts comprising a second absorbent capacity, wherein the second dry mass is no more than 15% greater than the first dry mass and the second absorbent capacity is at least 15% greater than the first absorbent capacity; and
one or more reusable outer covers configured to support each of the first absorbent inserts to form a first absorbent article and/or configured to support each of the second absorbent inserts to form a second absorbent article.

2. The array of claim 1 wherein the second absorbent capacity is at least 20% greater than the first absorbent capacity.

3. The array of claim 1 wherein the first dry mass is 25 g or less.

4. The array of claim 1 wherein the second dry mass is 30 g or less.

5. The array of claim 1 wherein the one or more reusable outer covers are disposed in a third package.

6. The array of claim 1 wherein at least one of the one or more reusable outer covers is disposed in the first package.

7. The array of claim 1 wherein at least one of the one or more reusable outer covers is disposed in the second package.

8. The array of claim 1 wherein the one or more reusable outer covers comprise a first reusable outer cover and a second reusable outer cover, wherein the first and second reusable outer cover differ by one of the group consisting of: graphics, color, size, material, and combinations thereof.

9. The array of claim 1 wherein each of the first absorbent inserts comprises a topsheet, a backsheet and an absorbent core disposed between the topsheet and the backsheet, and wherein the absorbent core is substantially free of cellulosic material.

10. The array of claim 1 wherein each of the second absorbent inserts comprises a topsheet, a backsheet and an absorbent core disposed between the topsheet and the backsheet, and wherein the absorbent core is substantially free of cellulosic material.

11. The array of claim 1 wherein each of the one or more reusable outer covers are configured to be used with each of the first absorbent inserts and configured to be used with each of the second absorbent inserts.

12. The array of claim 1 wherein each of the first and second absorbent inserts are disposable.

13. The array of claim 1 wherein each of the first and second absorbent inserts comprise a caliper of 20 mm or less.

14. The array of claim 1 wherein each of the first and second absorbent inserts comprise standing cuff systems.

15. The array of claim 1 wherein each of the first absorbent inserts comprises a first acquisition distribution system, and wherein each of the second absorbent inserts comprise a second acquisition distribution system.

16. The array of claim 15 wherein the first and second acquisition distribution systems differ by one of the group consisting of: ADS materials, basis weight, layer configuration, mass, caliper and combinations thereof.

17. The array of claim 1 wherein each of the first and second absorbent inserts comprise a rewet of 0.1 g or less.

18. The array of claim 1 wherein each of the first and second absorbent inserts comprise a wearer surface area of at least 475 cm².

19. The array of claim 1 wherein each of the first and second absorbent articles comprise at least 10% bio-based content.

20. A package of absorbent articles comprising:
a first absorbent insert comprising a first absorbent capacity and a first dry mass; and
a second absorbent insert comprising a second absorbent capacity and a second dry mass, wherein the second dry mass is no more than 15% greater than the first dry mass and the second absorbent capacity is at least 15% greater than the first absorbent capacity.

21. The package of claim 20 further comprising a reusable outer cover configured to support the first absorbent insert to form a first absorbent article and configured to support the second absorbent insert to form a second absorbent article.

22. The package of claim 20 wherein the second absorbent capacity is at least 20% greater than the first absorbent capacity.

23. The package of claim 20 wherein each of the first and second absorbent inserts comprise a rewet of 0.1 g or less.

24. The package of claim 20 wherein each of the first and second absorbent inserts comprise a wearer surface area of at least 475 cm².

25. The package of claim 20 wherein each of the first and second absorbent articles comprise at least 10% bio-based content.
